(19) United States
(12) Patent Application Publication
(10) Pub. No.: US 2024/0101721 A1
(43) Pub. Date: Mar. 28, 2024

(54) HYDROPHOBICALLY-MODIFIED POLYSACCHARIDES AND USES THEREOF

(71) Applicants: RHODIA OPERATIONS, Lyon (FR); SOLVAY USA INC., Princeton, NJ (US)

(72) Inventors: Ahmed Alsayed, Columbus, NJ (US); Manasi Chawathe, Bristol, PA (US); Celine Anna Simone Burel, Ploufragan (FR); Denis Bendejacq, Philadelphia, PA (US)

(73) Assignees: RHODIA OPERATIONS, Lyon, NJ (FR); SOLVAY USA INC., Princeton, NJ (US)

(21) Appl. No.: 17/767,832

(22) PCT Filed: Oct. 8, 2020
(86) PCT No.: PCT/US2020/054681

§ 371 Date: Apr. 8, 2022

Related U.S. Application Data
(60) Provisional application No. 62/912,248, filed on Oct. 8, 2019.
(60) Provisional application No. 62/966,176, filed on Jan. 27, 2020.

(51) Int. Cl. C08B 37/00; A61K 8/11; A61K 8/73; A61Q 13/00; B01J 13/08; B01J 13/20

(52) U.S. Cl. CPC C08B 37/0096; A61K 8/11; A61K 8/737; A61Q 13/00; B01J 13/08; B01J 13/20

(57) ABSTRACT

Described herein are hydrophobically-modified polysaccharides, their manufacture, and their use in microencapsulation, typically of water-insoluble active materials.

### CROSS REFERENCE TO RELATED APPLICATIONS

This application claims the priority of U.S. Provisional Applications No. 62/912,248, filed Oct. 8, 2019, and 62/966,176, filed Jan. 27, 2020, and both of which are hereby incorporated by reference in their entireties.

### FIELD OF THE INVENTION

The present disclosure relates to hydrophobically-modified polysaccharides, their manufacture, and their use in microencapsulation, typically of water-insoluble active materials.

### BACKGROUND

Microencapsulation finds use in the creation of products and applications within a large variety of industries, such as, for example, the pharmaceutical industry, cosmetics, household and personal care, food, agriculture, chemistry, and biotechnology, among others. Microencapsulation is a technique by which active, often sensitive, chemical compounds are entrapped in wall materials that protect them against adverse chemical and environmental reactions as well as loss, particularly in the case of volatile compounds. Microencapsulation of such active chemical compounds may also be used for the purpose of controlled release from the protected form. The release of the active chemical compound from the protected form may be rapid (such as by crushing, or by ingestion), or gradual (such as by dissolution, diffusion, or bio-degradation). In this manner it is possible to maximize the effectiveness of the active chemical compound by ensuring that it is released within the proper environment and time.

Synthetic polymers, such as Melamine Formaldehyde and Urea Formaldehyde, are typically used in microencapsulation. However, a major drawback of using such synthetic polymers, as in the case of Melamine Formaldehyde and Urea Formaldehyde, in microencapsulation is the health hazard presented by formaldehyde, which is known to be toxic and hazardous to human health. Another drawback of using synthetic polymers in microencapsulation is the propensity of the microcapsules, which are typically non-biodegradable, to enter the aquatic environment, since the filter of waste water treatment plants cannot remove the tiny particles. Such non-biodegradable particles pose an ecological danger. For example, once arrived in the oceans, algae can adhere and accumulate on the surface of the particles, thus entering the marine food chain.

Therefore, there is an ongoing need for materials suitable for use in microencapsulation that are environmentally-friendly and safe to handle.

### SUMMARY OF THE INVENTION

This objective, and others which will become apparent from the following detailed description, are met, in whole or in part, by the materials and processes of the present disclosure.

In a first aspect, the present disclosure relates to a process for synthesizing a hydrophobically-modified polysaccharide, the process comprising reacting a polysaccharide with a compound represented by formula (I)

G-R (I)

wherein
G is a carbonyl-containing functional group, and
R is a hydrophobic group.

In a second aspect, the present disclosure relates to a process for forming an emulsion, the process comprising mixing a hydrophobically-modified polysaccharide synthesized by the process described herein, typically in aqueous solution or dispersion, with a water-insoluble active material, thereby forming the emulsion.

In a third aspect, the present disclosure relates to a microcapsule comprising a core having a water-insoluble active material and a wall having the hydrophobically-modified polysaccharide synthesized by the process described herein.

In a fourth aspect, the present disclosure relates to a microcapsule comprising a core having a water-insoluble active material and a wall having a hydrophobically-modified polysaccharide, wherein the hydrophobically-modified polysaccharide comprises a polysaccharide having at least one hydrophobic substituent, wherein the hydrophobic substituent is a (C₅-C₄₅)ether, (C₅-C₄₅)ester, or mixtures thereof; typically a (C₅-C₂₇)ether, (C₅-C₂₇)ester, or mixtures thereof; more typically a (C₁₀-C₂₃)ether, (C₁₀-C₂₃)ester, or mixtures thereof.

### BRIEF DESCRIPTION OF THE FIGURES

FIG. 1 shows the emulsion size distribution as a function of time of emulsification using an Ultra Turaxx emulsifier.

FIG. 2 shows the emulsion size distribution as a function of aging time, the emulsion formed using an Ultra Turaxx emulsifier.

FIG. 3 shows the emulsion size distribution as a function of aging time, the emulsion formed using ultrasonicator.

FIG. 4 shows the percentage of weight retention of citronella oil in an inventive capsule in comparison to a comparative capsule and a control.

### DETAILED DESCRIPTION

As used herein, the terms “a”, “an”, or “the” means “one or more” or “at least one” and may be used interchangeably, unless otherwise stated.

As used herein, the term “comprises” includes “consists essentially of” and “consists of.” The term “comprising” includes “consisting essentially of” and “consisting of.”

Unless defined otherwise, all technical and scientific terms used herein have the same meaning as is commonly understood by one of skill in the art to which this specification pertains.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

As used herein, the terminology “(Cx-Cy)” in reference to an organic group, wherein x and y are each integers, means that the group may contain from x carbon atoms to y carbon atoms per group.

As used herein, the term “hydrocarbyl” means a monovalent radical formed by removing one hydrogen atom from a hydrocarbon, typically a (C₁-C₄₀) hydrocarbon. Hydrocarbyl groups may be straight, branched or cyclic, and may be saturated or unsaturated. Exemplary hydrocarbyl groups include, but are not limited to alkyl, alkenyl, alkynyl, aryl, arylalkyl, arylalkenyl, and arylalkynyl groups.

As used herein, the term “alkyl” means a monovalent straight or branched saturated hydrocarbon radical, more typically, a monovalent straight or branched saturated (C₁-C₄₀)hydrocarbon radical, such as, for example, methyl, ethyl, n-propyl, isopropyl, n-butyl, isobutyl, tert-butyl, hexyl, 2-ethylhexyl, octyl, hexadecyl, octadecyl, eicosyl, behenyl, tricontyl, and tetracontyl.

As used herein, the term “alkenyl” means a monovalent straight or branched unsaturated hydrocarbon radical, more typically, a monovalent straight or branched unsaturated (C₂-C₄₀)hydrocarbon radical, having one or more double bonds. Exemplary alkenyl groups include, but are not limited to vinyl, allyl, 1-propenyl, octenyl, dodecenyl, octadecenyl, palmitoleyl, oleyl, and erucyl.

As used herein, the term “alkynyl” means a monovalent straight or branched unsaturated hydrocarbon radical, more typically, a monovalent straight or branched unsaturated (C₂-C₄₀)hydrocarbon radical, having one or more triple bonds. Exemplary alkynyl groups include, but are not limited to ethynyl, propynyl, and propargyl groups.

As used herein, the term “aryl” means a monovalent unsaturated hydrocarbon radical containing one or more six-membered carbon rings in which the unsaturation may be represented by three conjugated double bonds. Aryl radicals include monocyclic aryl and polycyclic aryl. Polycyclic aryl refers to a monovalent unsaturated hydrocarbon radical containing more than one six-membered carbon ring in which the unsaturation may be represented by three conjugated double bonds wherein adjacent rings may be linked to each other by one or more bonds or divalent bridging groups or may be fused together. Examples of aryl radicals include, but are not limited to, phenyl, anthracenyl, naphthyl, phenanthrenyl, fluorenyl, and pyrenyl.

Alkyl, alkenyl, and alkynyl groups may be substituted by one or more aryl groups. As used herein, such groups are referred to as arylalkyl, arylalkenyl, and arylalkynyl groups, respectively.

Any substituent or radical described herein may optionally be substituted at one or more carbon atoms with one or more, same or different, substituents described herein. For instance, an alkyl group may be further substituted with an aryl group or another alkyl group. Any substituent or radical described herein may also optionally be substituted at one or more carbon atoms with one or more substituents selected from the group consisting of halogen, such as, for example, F, Cl, Br, and I; nitro (NO₂), cyano (CN), hydroxy (OH), alkoxy, and aryloxy.

As used herein, the term “alkoxy” means a monovalent radical denoted as —O-alkyl, wherein the alkyl group is as defined herein. Examples of alkoxy groups, include, but are not limited to, methoxy, ethoxy, n-propoxy, isopropoxy, n-butoxy, isobutoxy, and tert-butoxy.

As used herein, the term “aryloxy” means a monovalent radical denoted as —O-aryl, wherein the aryl group is as defined herein. Examples of aryloxy groups, include, but are not limited to, phenoxy, anthracenoxy, naphthoxy, phenanthrenoxy, and fluorenoxy.

In a first aspect, the present disclosure relates to a process for synthesizing a hydrophobically-modified polysaccharide, the process comprising reacting a polysaccharide with a compound represented by formula (I)

G-R (I)

wherein
G is a carbonyl-containing functional group, and
R is a hydrophobic group.

The G substituent of the compound represented by formula (I) is a carbonyl-containing functional group. In an embodiment, G comprises an anhydride group, an aldehyde group, an acid halide group, typically acid chloride, acid bromide, or acid iodide, a ketone group, or an amide group. In another embodiment, G is an anhydride group or an aldehyde group.

In an embodiment, G is an anhydride group represented by the structure

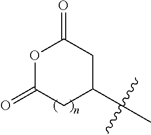

wherein n is 0 or 1, typically 0.

In another embodiment, G is an aldehyde group, typically represented as —(C═O)H.

The R substituent of the compound represented by formula (I) is a hydrophobic group. As would be known by those of ordinary skill in the art, hydrophobic means “water-hating” and refers to chemicals or chemical groups that repel water or are immiscible with water.

In an embodiment, R is hydrocarbyl, typically alkyl, alkenyl, alkynyl, aryl, arylalkyl, arylalkenyl, arylalkynyl, each being optionally substituted.

In another embodiment, R is (C₁-C₄₀)hydrocarbyl, typically (C₁-C₂₂)hydrocarbyl, more typically (C₆-C₁₈)hydrocarbyl.

In an embodiment, the compound represented by formula (I) is an alkenylsuccinic anhydride in which G is an anhydride group represented by the structure

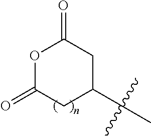

wherein n is 0; and R is an alkenyl group, typically (C₆-C₁a)alkenyl group.

In another embodiment, the compound represented by formula (I) is an arylalkenyl aldehyde, in which G is an aldehyde group, and R is an aryl group or an arylalkenyl group.

Compounds represented by formula (I) are available from commercial sources or may be synthesized according to methods known to persons of ordinary skill in the art.

Generally, polysaccharides are long chains of monosaccharides, such as, for example, fructose, galactose, glucose, mannose, xylose, arabinose, rhamnose, and stereoisomers and derivatives thereof, linked by glycosidic bonds.

Examples of polysaccharides suitable for use according to the present disclosure include, but are not limited to, glucan, starch, amylose, amylopectin, glycogen, dextran, cellulose, mannan, xylan, lignin, araban, galactan, galacturonan, chitin, chitosan, glucuronoxylan, arabinoxylan, xyloglucan, glucomannan, pectin, arabinogalactan, carrageenan, agar, gum arabic, gum tragacanth, ghatti gum, karaya gum, carob gum, polygalactomannan, or a mixture thereof. In an embodiment, the polysaccharide is a polygalactomannan. Polysaccharides suitable for use according to the present disclosure may be in an unmodified or in a derivatized form prior to reaction with the compound represented by formula (I). Polysaccharides in derivatized form prior to reaction with the compound represented by formula (I) include, but are not limited to, anionic, nonionic, and cationic polysaccharides, typically cationic polysaccharides.

Processes for making derivatives of polysaccharides are generally known. Typically, the polysaccharide is reacted with one or more derivatizing agents under appropriate reaction conditions to produce the corresponding polysaccharide having the desired substituent groups. Suitable derivatizing reagents are commercially available and typically contain a reactive functional group, such as an epoxy group, a chlorohydrin group, or an ethylenically unsaturated group, and at least one other substituent group, such as a cationic, nonionic or anionic substituent group, or a precursor of such a substituent group per molecule, wherein substituent group may be linked to the reactive functional group of the derivatizing agent by bivalent linking group, such as an alkylene or oxyalkylene group. Suitable cationic substituent groups include primary, secondary, or tertiary amino groups or quaternary ammonium, sulfonium, or phosphinium groups. Suitable nonionic substituent groups include hydroxyalkyl groups, such as hydroxypropyl groups. Suitable anionic groups include carboxyalkyl groups, such as carboxymethyl groups. The cationic, nonionic and/or anionic substituent groups may be introduced to the polysaccharide chains via a series of reactions or by simultaneous reactions with the respective appropriate derivatizing agents.

In an embodiment, the polysaccharide is in a derivatized form, typically a cationic polysaccharide. Such a cationic polysaccharide is obtainable by reacting the polysaccharide with a derivatizing agent having a cationic substituent group that comprises a cationic nitrogen radical, typically, a quaternary ammonium radical. Exemplary quaternary ammonium radicals are trialkylammonium radicals, such as trimethylammonium radicals, triethylammonium radicals, and tributylammonium radicals; aryldialkylammonium radicals, such as benzyldimethylammonium radicals; and ammonium radicals in which the nitrogen atom is a member of a ring structure, such as pyridinium radicals and imidazoline radicals, each in combination with a counterion, typically a chloride, bromide, or iodide counterion. In some embodiments, the cationic substituent group is linked to the reactive functional group of the derivatizing agent, for example, by an alkylene or oxyalkylene linking group. Derivatizing agents in which the cationic substituent group is linked to the reactive functional group by an alkylene or oxyalkylene linking group include, for example, epoxy-functional cationic nitrogen compounds, such as, 2,3-epoxypropyltrimethylammonium chloride; chlorohydrin-functional cationic nitrogen compounds, such as, 3-chloro-2-hydroxypropyl trimethylammonium chloride, 3-chloro-2-hydroxypropyl-lauryldimethylammonium chloride, 3-chloro-2-hydroxypropyl-stearyldimethylammonium chloride; and vinyl-, or (meth)acrylamide-functional nitrogen compounds, such as methacrylamidopropyl trimethylammonium chloride.

The degree of cationic substitution (DS_{cat}) of the cationic polysaccharide is generally from about 0.05 to 0.5, typically from about 0.05 and 0.3, more typically from about 0.05 and 0.2. As used herein, the term “degree of cationic substitution” refers to the average number of moles of cationic substitution per mole of sugar unit in the polysaccharide.

Polygalactomannans, or galactomannans, are polysaccharides consisting mainly of mannose and galactose. The mannose-elements form a chain consisting of many hundreds of (1,4)-3-D-mannopyranosyl-residues, with 1,6 linked-D-galactopyranosyl-residues at varying distances, dependent on the plant of origin. Naturally occurring galactomannans are available from numerous sources, including guar gum, guar splits, locust bean gum, flame tree gum and cassia gum. Additionally, galactomannans may also be obtained by classical synthetic routes or may be obtained by chemical modification of naturally occurring galactomannans. In an embodiment, the polygalactomannan is guar. In another embodiment, the polygalactomannan is cationic guar.

In accordance with the process described herein, reacting the polysaccharide with the compound represented by formula (I) may be conducted using any method known to those of ordinary skill in the art. For example, the polysaccharide may be dissolved in a first solution, typically aqueous solution, and the compound represented by formula (I) may be dissolved in a second solution, typically aqueous solution, after which the second solution is added to the first solution or, alternatively, the first solution is added to the second solution. The reaction may be subjected to mixing and/or agitation using any means known to those of ordinary skill, such as, for example, stirring or sonication.

The amount of the compound represented by formula (I) used in the reaction is not particularly limited. However, an amount of the compound represented by formula (I) from 1 to 99% by weight, typically 1 to 60%, more typically 5 to 50% by weight, relative the amount of the polysaccharide, is suitable.

The weight average molecular weight of the polysaccharide is suitably from 1,000 g/mol to 10,000,000 g/mol, typically 2,000 g/mol to 1,000,000 g/mol, more typically 3,000 g/mol to 500,000 g/mol, still more typically 5,000 g/mol to 100,000 g/mol. Generally, the weight average molecular weight may be measured by methods known to those of ordinary skill in the art, such as SEC-MALS (Size Exclusion Chromatography with detection by Multi-Angle Light-Scattering detection).

The pH of the reaction is not particularly limited. However, the pH of the reaction is typically greater than 7. In an embodiment, the pH is from 8 to 14, typically from 9 to 14. The pH may be adjusted according to methods known to those of ordinary skill in the art. For example, base may be added to the reaction mixture. Suitable bases include sources of hydroxide (—OH), for example, alkali metal hydroxides, alkaline earth metal hydroxides, and mixtures thereof. Exemplary alkali metal hydroxides include, but are not limited to, LiOH, NaOH, KOH, RbOH, and CsOH. Exemplary alkaline earth metal hydroxides include, but are not limited to, Mg(OH)₂, Ca(OH)₂, Sr(OH)₂, and Ba(OH)₂.

The hydrophobically-modified polysaccharide made according to the process described herein may be characterized by its degree of hydrophobic substitution. As used herein, the terminology “degree of hydrophobic substitution” refers to the average number of hydrophobic groups attached to each monomeric unit of the polysaccharide as a result of the reaction between the polysaccharide and the compound represented by formula (I). The degree of substitution may be determined using methods known to those of ordinary skill in the art, for example, by ¹H NMR. In an embodiment, the degree of substitution (DSh) of the hydrophobically-modified polysaccharide synthesized is from about 0.001 to about 3.0, more typically from about 0.002 to about 2.5, still more typically from about 0.004 to about 0.5.

The hydrophobically-modified polysaccharide made according to the process described herein has been found to be very suitable as an emulsifying agent for water-insoluble materials, forming very stable emulsions with such materials.

Thus, in a second aspect, the present disclosure relates to a process for forming an emulsion. Formation of the emulsion is achieved by mixing a hydrophobically-modified polysaccharide synthesized by the process described herein, typically in aqueous solution or dispersion, with a water-insoluble material, typically a water-insoluble active material.

Mixing of the hydrophobically-modified polysaccharide with the water-insoluble material may be conducted according to any known method. For example, in one suitable method, the hydrophobically-modified polysaccharide is dispersed in an aqueous medium and the water-insoluble material is subsequently added. The resulting mixture may be subjected to mixing and/or agitation using any means known to those of ordinary skill in the art, such as, for example, stirring, sonication, or high shear treatment.

Herein, “aqueous medium” refers to a medium comprising or consisting of water. The aqueous medium may comprise one or more organic solvents that are miscible in water. Such organic solvents that are miscible in water include, but are not limited to, alcohols, such as methanol, ethanol, propanol, and the like; amines, such as ethylamine, diethanolamine, and the like; ketones, such as acetone; and amides, such as dimethylformamide (DMF). Other organic solvents that are miscible in water include, but are not limited to, dimethyl sulfoxide (DMSO), pyridine, tetrahydrofuran (THF), and acetonitrile.

The water-insoluble material suitable for use in the emulsion described herein is generally an oil that is not soluble in aqueous medium and may be an active material. Herein, “water-insoluble active material” refers to a material that provides a benefit to a user. A water-insoluble active material may comprise, for example, a flavor, fragrance, pro-fragrance, taste masking agent, taste sensate, malodor counteracting agent, vitamin, antibacterial agent, sunscreen active, antioxidant, anti-inflammatory agent, anesthetic, analgesic, antifungal agent, antibiotic, anti-viral agents, anti-parasitic agent, anti-infectious agent, anti-acne agent, dermatological active ingredient, enzymes and co-enzymes, skin whitening agent, anti-histamine, chemotherapeutic agent, insect repellent, or a mixture thereof.

Other optional active materials useful in the pharmaceutical, cosmetics, household and personal care, food, agriculture, chemistry, and biotechnology industries may also be used as long as the optional active materials are water-insoluble. Such optional active materials are most typically those described in reference books, such as the CTFA Cosmetic Ingredient Handbook, Second Edition, The Cosmetic, Toiletries, and Fragrance Association, Inc. 1988, 1992; and Handbook of Flavors and Fragrances by M. and I. Ash, Synapse Information Resources Inc., 2006.

Generally, as would be understood by those of ordinary skill in the art, emulsions are characterized by a dispersed phase and a continuous phase, in which the dispersed phase typically exists as droplets within the continuous phase. The droplets of dispersed phase may optionally be encapsulated by an emulsifying agent, which acts to prevent coalescence of the dispersed phase and stabilize the emulsion. It has been found that the hydrophobically-modified polysaccharide of the present disclosure acts as a very good emulsifying agent for water-insoluble material and an emulsion comprising the said components remain stable for long periods of time.

The size of the dispersed phase droplets, herein referred to as emulsion droplet size, is may be determined by dynamic light scattering using standard instrumentation. In the emulsion described herein, the emulsion droplet size is typically from 0.1 μm to 200 μm.

The emulsion made by mixing the hydrophobically-modified polysaccharide and the water-insoluble material remains stable for long periods of time. In an embodiment, the emulsion formed is stable for at least 1 week, at least 2 weeks, at least 3 weeks, at least 5 weeks, at least 8 weeks, or at least 12 weeks, or at least 18 weeks. In another embodiment, the emulsion formed is stable for at least 30 months.

The emulsion described herein may be further reacted with a crosslinker to crosslink the hydrophobically-modified polysaccharide, for example, to further stabilize the emulsion. Suitable crosslinkers include, but are not limited, glyoxal; phenolic di-aldehydes, such as terephthalaldehyde or di-vanillin; borates, such as sodium tetraborate; and metal containing compounds, such as titanium- and zirconium-containing compounds. Such crosslinkers are typically available from commercial sources or synthesized according to methods well-known to those of ordinary skill.

Titanium-containing crosslinkers include, but are not limited to, titanium salts, such as titanium tetrachloride, titanium tetrabromide, or tetra amino titanate; titanium chelates, such as titanium acetylacetonates, triethanolamine titanates, and titanium lactates; titanium esters such as n-butyl polytitanates, titanium tetrapropanolate, octyleneglycol titanates, tetra-n-butyl titanates, tetra-n-buytl titanates, tetra-2-ethylhexyl titanates, tetra-isopropyl titanate, and tetra-isopropyl titanate; and mixtures thereof.

Suitable zirconium-containing crosslinkers include, but are not limited to, zirconium salts, such as zirconium citrate, zirconium tartate, and zirconium glycolate; zirconium chelates, such as zirconium acetylacetonates, di- or tri-ethanolamine zirconates, and zirconium lactates; and mixtures thereof. Suitable zirconium lactates include, but are not limited to, zirconium ammonium lactate, zirconium di- or tri-ethanolamine lactate, zirconium diisopropylamine lactate, and zirconium sodium lactate salts.

Other crosslinkers suitable for use according to the present disclosure include salts comprising at least one multivalent cation, polyalkoxysiloxanes, and the like. As used herein, salts comprising at least one multivalent cation are salts in which at least one cation has an oxidation state of 2+ or higher. Suitable salts include, but are not limited to, salts comprising one or more cations selected from the group consisting of Group 2 (IUPAC numbering) metal cations, such as Mg²⁺, Ca²⁺, Sr²⁺, and Ba²⁺, Group 13 metal cations, such as Al³⁺, and transition metal cations, such as Fe 3+. The anion or anions of such suitable salts are not particularly limited and may include halides, such as F⁻, Cl⁻, Br⁻, I⁻, nitrates, sulfates, and phosphates. In an embodiment, the crosslinker is a salt comprising at least one multivalent cation, typically Ca²⁺. In an embodiment, the crosslinker is CaCl₂).

Polyalkoxysiloxanes are partially condensed products of tetraalkoxysilanes. The alkoxy groups in suitable polyalkoxysiloxanes may be identical or different. Polyalkoxysiloxanes may or may not be hyperbranched. Exemplary polyalkoxysiloxanes include, but are not limited to, polymethoxysiloxanes, polyethoxysiloxanes, polypropoxysiloxanes, and polybutoxysiloxanes. Polyalkoxysiloxanes may be obtained from commercial sources or may be synthesized according to known procedures (Abe, Y.; Shimano, R.; Arimitsu, K.; Gunji, T., Preparation and Properties of High Molecular Weight Polyethoxysiloxanes Stable to Self-Condensation by Acid-Catalyzed Hydrolytic Polycondensation of Tetraethoxysilane. J. Polym. Sci., Part A: Polym. Chem. 2003, 41, 2250-2255, DE 10261289 A1 or Zhu, X. M.; Jaumann, M.; Peter, K.; Moller, M.; Melian, C.; Adams-Buda, A.; Demco, D. E.; Blumich, B., One-Pot Synthesis of Hyperbranched Polyethoxysiloxanes. Macromolecules 2006, 39, 1701-1708). In an embodiment, the crosslinker is a polyalkoxysiloxane, typically polyethoxysiloxane (PEOS), more typically hyperbranched polyethoxysiloxane.

When the polysaccharide is a cationic polysaccharide, other crosslinkers are also suitable, such as multivalent anions. Exemplary multivalent anions include, but are not limited to carbonate, sulfate, citrate, and phosphate anions. The counterions and the number thereof for such multivalent anions are not particularly limited, but may include one or more alkali metal cations, such as sodium.

Without wishing to be bound by theory, it is believed that the droplets of dispersed phase in the continuous phase of the emulsion described herein are encapsulated by the hydrophobically-modified polysaccharide, which acts not only as an emulsifying agent to prevent coalescence of the dispersed phase but also wall material of a microcapsule.

Thus, in a third aspect, the present disclosure relates to a microcapsule comprising a core having a water-insoluble active material and a wall having the hydrophobically-modified polysaccharide synthesized by the process described herein.

The size of the microcapsules made by reacting the emulsion described herein with a crosslinker may be determined by dynamic light scattering using standard instrumentation, such as a Zetasizer instrument. In an embodiment, the microcapsule size is from 100 nm to 200 μm, typically from 200 nm to 100 μm, more typically from 300 nm to 30 μm.

In a fourth aspect, the present disclosure relates to a microcapsule comprising a core having a water-insoluble active material and a wall having a hydrophobically-modified polysaccharide, wherein the hydrophobically-modified polysaccharide comprises a polysaccharide having at least one hydrophobic substituent, wherein the hydrophobically-modified polysaccharide comprises a polysaccharide having at least one hydrophobic substituent, wherein the hydrophobic substituent is a (C₅-C₄₅)ether, (C₅-C₄₅)ester, or mixtures thereof; typically a (C₅-C₂₇)ether, (C₅-C₂₇)ester, or mixtures thereof; more typically a (C₁₀-C₂₃)ether, (C₁₀-C₂₃)ester, or mixtures thereof. In this respect, the hydrophobically-modified polysaccharide is typically formed from the process of reacting a polysaccharide, typically the polysaccharides described herein, with a compound represented by formula (I). In this respect, the hydrophobic substituent of the hydrophobically-modified polysaccharide is formed from the compound represented by formula (I).

In certain embodiments, the hydrophobic substituent, which is a (C₅-C₄₅)ether, (C₅-C₄₅)ester, or mixtures thereof; typically a (C₅-C₂₇)ether, (C₅-C₂₇)ester, or mixtures thereof; more typically a (C₁₀-C₂₃)ether, (C₁₀-C₂₃)ester, or mixtures thereof, comprises a (C₁-C₄₀)hydrocarbyl, typically (C₁-C₂₂)hydrocarbyl, more typically (C₆-Cis)hydrocarbyl. In other embodiments, the hydrophobic substituent of the hydrophobically-modified polysaccharide comprises at least one pendant carboxyl group.

In yet another aspect, the present disclosure relates to an emulsion comprising the microcapsules described herein, wherein 75% to 100%, typically 80% to 90%, of the microcapsules have a size from 100 nm to 200 μm, typically from 200 nm to 100 μm, more typically from 300 nm to 30 μm.

The processes and products according to the present disclosure are further illustrated by the following non-limiting examples.

### Example 1. Synthesis of Hydrophobically-Modified Polysaccharide Using Alkenyl Succinic Anhydrides

Guar (MW=25,000 g/mol), or cationic modified guar (Mw=25,000 g/mol with quaternary ammonium function with degree of susbstitution 0.12) was dissolved in water at a concentration of 10%. Octenyl succinic anhydride (OSA) was added to the solution of guar at concentration of 5, 10, 20 or 50 wt %, relative to the weight of guar. The reaction was carried out at 30° C. and pH was maintained at about 9 using 5% NaOH. The reaction was terminated by lowering the pH to 6 using 5% acetic acid. The polymer was purified by flocculation in methanol and isopropanol. The flocculated polymer was dispersed in water and dialyzed against a membrane of 3.5 kDa cut-off.

The same procedure was used to prepare hydrophobically-modified polysaccharide using guar and dodecenyl succinic anhydride (DSA), except that the reaction was carried out at 45° C. The same procedure was also used to prepare hydrophobically-modified polysaccharide using guar octadecenyl succinic anhydride (ODSA), except that the reaction was carried out at 60° C.

Hereinafter, the various hydrophobically-modified polysaccharide derivatives of the present example will be referred to as “guar/xSA(y %)”, wherein x represents 0, D, or OD depending on the alkyl succinic anhydride used and y is the weight percentage of alkyl succinic anhydride relative to the guar content.

### Example 2. Synthesis of Hydrophobically-Modified Polysaccharide Using Arylalkenyl Aldehydes

5 g guar (MW=25,000 g/mol) was dissolved in 45 g DI water. The pH of the guar solution was adjusted to above 13 using NaOH and the temperature was adjusted to 70° C. 2 g of cinnamaldehyde was added and the reaction was carried out for 2 hours.

### Example 3. Stability of Emulsion Formed by Sonication

Emulsions each containing 3.3 g hydrophobically-modified polysaccharide, 29.7 g water-insoluble oil, and 67 g water, by weight relative to the weight of the emulsion, were prepared as follows.

Each of the hydrophobically-modified polysaccharides synthesized according to Example 1 was dispersed in water. Water-insoluble oil (caprylic/capric triglyceride, marketed as NEOBEE® M-5 by Stepan) was added to each aqueous dispersion and the resulting mixtures were each sonicated using a probe sonicator for about 1 minute.

To determine the stability of the emulsions, samples of each emulsion were taken at weekly intervals over a period of at least 18 weeks and observed using optical microscopy. The samples were each diluted 10-fold and pictures were taken using an optical microscope under 100× magnification and processed by ImageJ software. The time at which the presence of oil separation and/or coalescence, which are indicative of lack of stability, was observed was noted. The results are summarized in Table 1 below.

TABLE 1

| Hydrophobically- | | % Alkenyl- | | |
|---|---|---|---|---|
| modified | Short | succinic | Stability | |
| polysaccharide | name | anhydride | period | |
| Guar/OSA | Guar/OSA(5%) | 5 | —* | |
| | Guar/OSA(10%) | 10 | —* | |
| | Guar/OSA(20%) | 20 | 8 | weeks |
| | Guar/OSA(50%) | 50 | 18 | weeks |
| Cationic guar/OSA | Cat. Guar/OSA(50%) | 50 | 2 | weeks |
| Guar/DSA | Guar/DSA(5%) | 5 | 2 | weeks |
| | Guar/DSA(10%) | 10 | 2 | weeks |
| | Guar/DSA(20%) | 20 | 5 | weeks |
| | Guar/DSA(50%) | 50 | 5 | weeks |
| Guar/ODSA | Guar/ODSA(5%) | 5 | 1 | week |
| | Guar/ODSA(20%) | 20 | 3 | weeks |
| | Guar/OSDA(50%) | 50 | 3 | weeks |

*less than 1 week

### Example 4. Emulsion Formed by High Shear

The effect of homogenization at 20,000 rpm using a homogenizer (ULTRA-TURRAX® homogenizer available from IKA) was investigated. An emulsion was formed by combining guar/OSA made from guar and OSA at concentration 50 wt %, relative to the weight of guar, as in Example 1 (herein referred to as “guar/OSA(50%)”) and water-insoluble oil (NEOBEE® M-5). The guar/OSA(50%) and water-insoluble oil was sheared at 20,000 rpm and aliquots were taken at 1, 5, and 15 minutes for analysis.

In one trial, following shearing, the emulsion was freeze-dried and resuspended at 30%.

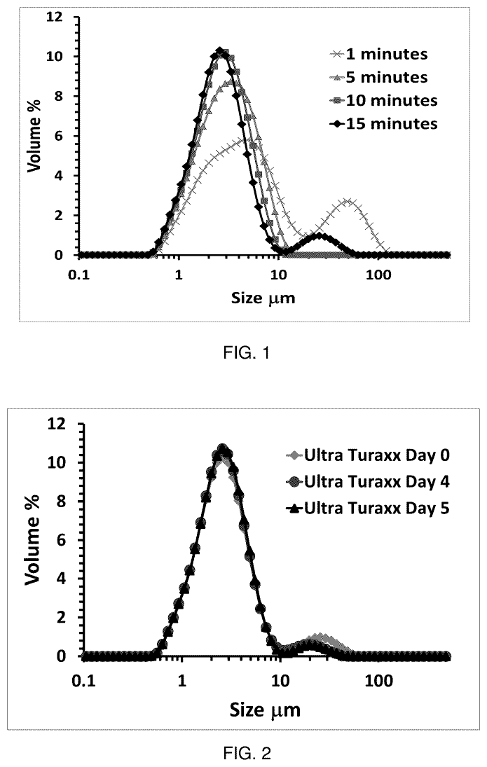

The emulsion droplet size for each sample was determined within the first 15 minutes of the preparation using a Mastersizer 3000 (Malvern), a Zetasizer analyzer (Malvern) in DLS mode as well as optical microscopy with image processing by ImageJ software. The emulsion droplet sizes are summarized in Table 2 below, and the size distributions are shown in FIG. 1 and FIG. 2.

TABLE 2

| | | | Image | Mastersizer | Mastersizer |
|---|---|---|---|---|---|
| | | Zetasizer | Analysis | 1^{st} Peak | 2^{nd} Peak |
| Time | | Size (nm) | | | |
| 1 | min | 3416 | 2200 | 4885 | 48619 |
| 5 | min | 2277 | 1700 | 3330 | — |
| 10 | min | 1504 | — | 2931 | — |
| 15 | min | 1067 | 1300 | 2580 | 22602 |
| 4 | days | — | — | 2580 | 19893 |
| 5 | days | — | — | 2580 | 19893 |

| | | | | |
|---|---|---|---|---|
| Resuspended | 785 | — | — | — |
| after freeze- | | | | |
| drying | | | | |

As shown in Table 2, the size does not evolve over the first 15 minutes of the preparation. The size was followed over 6 months afterwards, and no significant evolution was observed upon either visual, microscopic or Zetasizer inspection. The inventive emulsions thus show long-term stability.

### Example 5. Fragrance Emulsions

Emulsions each containing hydrophobically-modified polysaccharide guar/DSA(20%), water and different fragrance oils, were prepared as follows. 3.3 g of guar/DSA(20%) made from guar and DSA at concentration 20 wt %, relative to the weight of guar, as in Example 1 (herein referred to as “guar/DSA(20%)”) was dispersed in 67 g of water. 29.7 g of various fragrance oils (patchouli oil, citronella oil, or lemon oil) were added and the resulting mixtures were each sonicated using a probe sonicator for about 1 minute. All emulsions formed were stable upon both visual and microscopic inspection.

### Example 6. Fragrance Emulsion

0.8 g geraniol fragrance added to 10 g of a 5% solution of guar-cinnamaldehyde made in accordance with Example 2 and then sonicated for 1 minute. The droplet size was observed to be less than 5 μm. The emulsion formed was stable upon both visual and microscopic inspection.

### Example 7. Crosslinking of Emulsions—Formation of Capsules

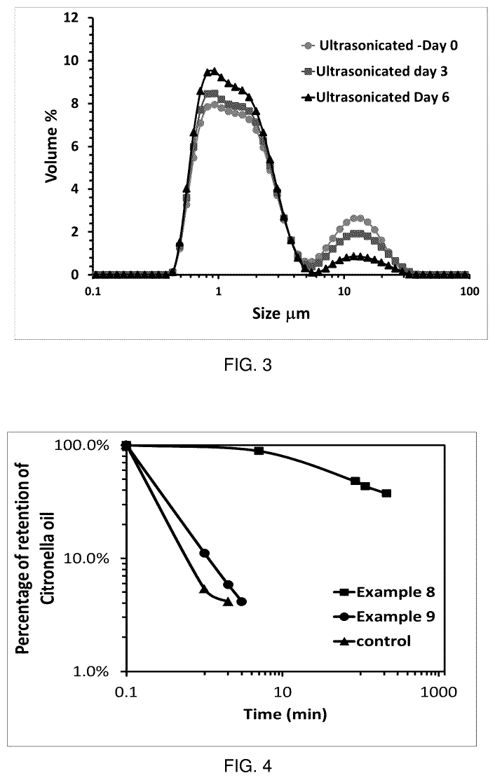

Guar/OSA(50%) was dispersed in water to which NEOBEE® M-5 was added as in Example 3. The resulting mixture was sonicated using a probe sonicator for about 1 minute. Subsequently, triethanolamine zirconate (5 wt % relative to the weight of guar/OSA(50%) used) was added with constant stirring. For comparison, an emulsion made according to the same procedure but without the crosslinking step was used as control. The emulsion droplet size for the control emulsion and the crosslinked emulsion was analyzed and the results summarized in Table 3 below, and the size distribution is shown in FIG. 3.

TABLE 3

| | | |
|---|---|---|
| | Mastersizer | Mastersizer |
| Zetasizer (DLS) Analysis | 1^{st} Peak | 2^{nd} Peak |

| Day | Size (nm) | | |
|---|---|---|---|
| 0 | 360 | 1055 | 13564 |
| 1 | 385 | | 13564 |
| 3 | 444 | 1055 | |
| 5 | 604 | 1055 | 13564 |
| 5 (crosslinked | 520 | | |
| 5%) | | | |

All emulsions formed were stable upon both visual and microscopic inspection.

### Example 8. Formation of Capsules

1 g of GA/OSA(50%) was dispersed in 90 mL of water. To 60 mL of this dispersion, 9 g of citronella oil (Sigma Aldrich, used as received) was added. Emulsion was made by an ULTRA-TURRAX® at 20 000 rpm, for 10 min. 0.816 g of CaCl₂) was dissolved in 10 mL water. This was added to the emulsion while stirring. After 1 h of stirring 30 mL of the GA-OSA dispersion in water was added to the emulsion. The average size of the objects formed was determined to be 2 micrometers.

### Comparative Example 9. Comparative Emulsion

1 g of GA/OSA(50%) was dispersed in 90 mL of water. To this dispersion, 9 g of citronella oil was added. Emulsion was made by an ULTRA-TURRAX®at 20 000 rpm, for 10 min.

### Example 10. Weight Retention Study

The retention of water-insoluble oil (citronella oil) by the inventive capsules over time was evaluated as follows.

4 glass substrates, 2×3 squared inch, were thoroughly cleaned with water and soap, then dried with nitrogen flux. For reference, the neat substrates were weighed. They were then coated with 0.25 g of the composition of Example 8 and Comparative Example 9, and dried at 140° C. for 1 min, then put in a dessicator for 12 h to remove the final traces of water. The glass substrates were then placed on a hot plate kept at 140° C. and weighed over time to follow the evaporation of citronella oil.

A non-emulsified/non-encapsulated composition, referred to as “control” in instant example, was also prepared by spreading 0.025 g citronella oil alone directly onto a fifth glass substrate, and its weigh was followed over time at 140° C. as well.

All weighing was carried out using a Sartorius® balance with 10 microgram accuracy. All samples were cooled down to room temperature before being weighed and heated again. The percentage of weight retention was calculated as the percentage of mass retained from the starting reference weight. The results are shown in FIG. 4.

As shown in FIG. 4, the control shows retention at 140° C. of 4% in 2 minutes. The composition of Example 8 shows 88% retention after 5 min at 140° C., and 38% after 215 min at 140° C., while the composition of Comparative Example 9 shows 4% retention after 3 min at 140° C.

### Example 11. Formation of Capsules

0.1 g of GA/OSA(50%) was dispersed in 9 mL of water. To 6 mL of this dispersion, 0.9 g of citronella oil was added. Emulsion was made by ultrasonication @ 10% amplitude for 1 min. 0.0816 g of CaCl₂) was dissolved in 1 mL water. This was added to the emulsion while stirring. After 1 h of stirring 3 mL of the GA-OSA dispersion in water was added to the emulsion.

### Example 12. Release Kinetics Study

The method described in Example 10 was used to follow the kinetics of release at 140° C. of composition of Example 11, prepared with ultrasonication, so as to compare it to the composition of Example 8, prepared with ULTRA-TURRAX®. The average size of the objects formed in composition of Example 12 is 0.5 micrometers.

After a few hours at 140° C., the composition of Example 11 showed a percentage of weight retention of 58%, which was higher than the 38% retention of the composition of Example 8.

### Example 13. Formation of Capsules

0.1 g of hyperbranched polyethoxysiloxane (PEOS, available from Scientific Polymer Products, Inc.) and 0.001 g of Nile red (Sigma Aldrich), were dissolved in 9 g of citronella oil. 0.9 g of GA/OSA(50%) was dispersed in 90 mL of water. To this dispersion, the 9 g of the PEOS/citronella oil/Nile red mixture was added. Emulsion was made by an ULTRA-TURRAX® at 20 000 rpm, for 10 min. pH was adjusted to 4 to 5, by using hydrochloric acid solution, as needed. The emulsion was stirred at room temperature for 1 to 5 days.

After drying at room temperature, no leaking of the citronella oil cargo from the formed capsules was observed by fluorescence microscopy after up to 5 days. The capsules were also redispersible in water and showed no change in size.

1. A process for synthesizing a hydrophobically-modified polysaccharide, the process comprising reacting a polysaccharide with a compound represented by formula (I)
G-R (I)
wherein
G is a carbonyl-containing functional group, and
R is a hydrophobic group.

2. The process according to claim 1, wherein G comprises an anhydride group, an aldehyde group, an acid halide group, a ketone group, or an amide group.

3. The process according to claim 1, wherein G is an anhydride group represented by the structure 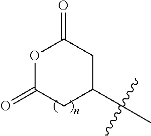
wherein n is 0 or 1.

4. The process according to claim 1, wherein G is an aldehyde group.

5. The process according to claim 1, wherein R is a hydrocarbyl selected from the group consisting of alkyl, alkenyl, alkynyl, aryl, arylalkyl, arylalkenyl, and arylalkynyl, each being optionally substituted.

6. The process according to claim 1, wherein R is (C₁-C₄₀)hydrocarbyl.

7. The process according to claim 1, wherein the polysaccharide is a glucan, starch, amylose, amylopectin, glycogen, dextran, cellulose, mannan, xylan, lignin, araban, galactan, galacturonan, chitin, chitosan, glucuronoxylan, arabinoxylan, xyloglucan, glucomannan, pectin, arabinogalactan, carrageenan, agar, gum arabic, gum tragacanth, ghatti gum, karaya gum, carob gum, polygalactomannan, or a mixture thereof.

8. The process according to claim 1, wherein a degree of hydrophobic substitution (DS h) of the hydrophobically-modified polysaccharide is from 0.001 to 3.0.

9. The process according to claim 1, wherein an amount of the compound represented by formula (I) is from 1 to 99% by weight relative the amount of the polysaccharide.

10. The process according to claim 1, wherein the weight average molecular weight of the polysaccharide is from 1,000 g/mol to 10,000,000 g/mol.

11. A hydrophobically-modified polysaccharide synthesized by the process according to claim 1.

12. A process for forming an emulsion, the process comprising mixing the hydrophobically-modified polysaccharide according to claim 11, in aqueous solution or dispersion; with a water-insoluble active material, thereby forming the emulsion.

13. The process according to claim 12, wherein the water-insoluble active material comprises a flavor, fragrance, pro-fragrance, taste masking agent, taste sensate, malodor counteracting agent, vitamin, antibacterial agent, sunscreen active, antioxidant, anti-inflammatory agent, anesthetic, analgesic, antifungal agent, antibiotic, anti-viral agents, anti-parasitic agent, anti-infectious agent, anti-acne agent, dermatological active ingredient, enzymes and co-enzymes, skin whitening agent, anti-histamine, chemotherapeutic agent, insect repellent, or a mixture thereof.

14. The process according to claim 12, wherein the emulsion comprises discrete droplets having a droplet size from 0.1 μm to 200 μm.

15. The process according to claim 12, wherein the emulsion is further reacted with a crosslinker.

16. The process according to claim 12, wherein the emulsion formed is stable for at least 1 week.

17. The process according to claim 12, wherein the emulsion comprises one or more microcapsules, wherein the one or more microcapsules each comprise a core having the water-insoluble active material and a wall having the hydrophobically-modified polysaccharide.

18. An emulsion formed according to the process of claim 12.

19. A microcapsule comprising a core having a water-insoluble active material and a wall having the hydrophobically-modified polysaccharide according to claim 11.

20. The microcapsule according to claim 19, wherein the microcapsule has a size from 100 nm to 50 μm.

21. The process according to claim 15, wherein the crosslinker is a salt comprising at least one multivalent cation.

22. The process according to claim 21, wherein the crosslinker is CaCl₂.

23. The process according to claim 15, wherein the crosslinker is a polyalkoxysiloxane.

24. A microcapsule comprising a core having a water-insoluble active material and a wall having a hydrophobically-modified polysaccharide, wherein the hydrophobically-modified polysaccharide comprises a polysaccharide having at least one hydrophobic substituent, wherein the hydrophobic substituent is a (C₅-C₄₅)ether, (C₅-C₄₅)ester, or a mixture thereof.

25. The microcapsule according to claim 24, wherein the at least one hydrophobic substituent comprises a (C₁-C₄₀)hydrocarbyl.

26. The microcapsule according to claim 24, wherein the at least one hydrophobic substituent comprises at least one pendant carboxyl group.

27. The microcapsule according to claim 24, wherein the microcapsule has a size from 100 nm to 200 μm.

28. The microcapsule according to claim 24, wherein the polysaccharide is selected from the group consisting of a glucan, starch, amylose, amylopectin, glycogen, dextran, cellulose, mannan, xylan, lignin, araban, galactan, galacturonan, chitin, chitosan, glucuronoxylan, arabinoxylan, xyloglucan, glucomannan, pectin, arabinogalactan, carrageenan, agar, gum arabic, gum tragacanth, ghatti gum, karaya gum, carob gum, polygalactomannan, and combinations thereof.

29. An emulsion comprising a plurality of the microcapsule according to claim 24, wherein 75% to 100% of the microcapsules have a size from 100 nm to 200 μm.
